(19) United States
(12) Patent Application Publication
(10) Pub. No.: US 2020/0025228 A1
(43) Pub. Date: Jan. 23, 2020

(54) Strap with semi-flat spring rate

(71) Applicant: Bionic Power Inc., Vancouver (CA)

(72) Inventors: Charles David Halliburton, Port Moody (CA); Daniel Loren Hepler, Vancouver (CA); Clive Edward Mullins, North Vancouver (CA)

(21) Appl. No.: 16/181,545

(22) Filed: Nov. 6, 2018

Related U.S. Application Data
(60) Provisional application No. 62/699,144, filed on Jul. 17, 2018.

(51) Int. Cl. F16B 2/08; F16M 13/04

(52) U.S. Cl. CPC F16B 2/08; F16M 13/04

(57) ABSTRACT

The length of a strap has an inelastic component and a stretchable component. The stretchable component has a flat, nearly flat or degressive spring rate for the majority of its operating range of extension. The operating range accommodates the expansion and contraction of a body part to which the strap is to be attached, such that the tension applied to the body part by the strap varies minimally as the body part expands and contracts, increasing comfort for the wearer.

### TECHNICAL FIELD

This application relates to straps that fasten to a human. More specifically, it relates to a system for alleviating the tension on an expanding human body part, produced by wearing a strap, by the use of a flat, nearly flat or degressive spring rate to control the tension.

### BACKGROUND

Most objects or devices that are meant to be carried or worn use elastic straps in order to be coupled with the body, which may be a human, animal or robot body. One challenge raised using this type of strap is that the elastic nature of the strap causes the displacement of the object or device over time. A functional device, which is required to be held in an optimum position on the body, gradually moves away from its initial position as the strap loses its ability to provide an efficient coupling.

Braces, prosthetics and other devices are required to couple tightly to the human, animal, or robot overlay body, for example to the legs, waist, arms, torso or feet. To function efficiently, displacement of the device must be minimized and the coupling should be stiff.

Current devices use elastic straps. Normally, an elastic strap exhibits a linear spring rate behavior, wherein the extension of the strap is proportional to the applied force or tension, or progressive, where the spring rate increases with extension. For example, the inner rubber of an elastic strap is linear but the webbing is somewhat rigid. The combination of these two makes for a combined spring rate curve, albeit mostly linear.

In some applications, such as energy harvesting devices strapped around a user's leg, it is required that the straps be reasonably tight in order to achieve good coupling of the harvester to the leg. During normal walking, an elastic strap is comfortable. However, when the user bends beyond the normal walking range of motion, for example when squatting, the leg circumference grows. The strap is then forced to extend, and as an elastic strap has a linear or progressive spring rate, the strap tension increases, creating additional pressure on the user resulting in discomfort. This problem is common for medical devices, backpacks, harnesses, clothing, braces, utility belts or any medical device that should be attached with a strap. Additionally, there are safety implications: if the strap or device becomes snagged, the stress on the strap may rise until something breaks, which may also occur when, for example, a movement causes the maximum extension of the strap to be exceeded.

Applications where non-linear spring rates are used include Belleville washers, for example. Other applications include leaf springs where a force is applied at an acute angle to the leaf. Lever linkages that have progressive ratios are found in bicycles. Nautilus or other non-circular gears have changing ratios that can be degressive or progressive. Compound bows use cams with a changing pulley arm to achieve a degressive spring rate, meaning that it is initially hard to pull the bow but when it is fully drawn it requires a small holding force to hold it.

This background is not intended, nor should be construed, to constitute prior art against the present invention.

### SUMMARY OF INVENTION

The present invention is directed to a strap that incorporates an inelastic component and a stretchable component. The stretchable component is a mechanism with a semi-flat spring rate (flat, nearly flat or degressive) for the majority of its operating range of extension. The semi-flat spring rate may be the result of a combination of several mechanisms that exhibit various different spring rate profiles such as flat, degressive, constant or linear spring rates. The operating range accommodates the expansion and contraction of a body part to which the strap is to be attached, such that the tension applied to the body part by the strap varies minimally as the body part expands and contracts.

Different embodiments of the invention are presented here in order to illustrate the practicality of the incorporation of a semi-flat spring rate profile into a strap. Embodiments include straps with a cam mechanism and inelastic band; a stretchable band and inelastic buckle; and a flat coil spring mechanism and inelastic band.

For the reasons of comfort, the coupling must be able to allow for expansion of the body as the wearer moves, without imparting significant extra tension. The advantages of each embodiment of the present invention include one or more of: helping to mitigate excessive pressure applied to a person who is wearing the strap; improving coupling of a device to a person; and improving power transmission between a device and the human body.

A practical use of the strap is to connect a genuflective energy harvester to a person. Such an energy harvester is connected between a shin brace and a thigh brace attached to a person's leg. The braces change angle relative to each other as the person locomotes, generating electricity in the harvester. It is important for the braces to be connected firmly yet comfortably to the person for maximum efficiency and effectiveness of the energy harvester. Straps are either used to attach through holes or slots in the braces, or they are incorporated as components of the braces.

As disclosed, an aspect of the present invention is a strap for tightening around an expandable object comprising: a first portion that is inelastic; and a second portion connected to the first portion and having a spring rate profile that is semi-flat in an operating range spanning a first extension of the second portion and a second extension of the second portion; wherein, when the strap is tightened around the expandable object, the second portion remains within the operating range as the expandable object expands and contracts.

### BRIEF DESCRIPTION OF DRAWINGS

The following drawings illustrate embodiments of the invention and should not be construed as restricting the scope of the invention in any way.

FIG. 1 is a schematic representation of a strap, according to an embodiment of the present invention.

FIG. 2 is a graph showing a range of various spring rate profiles in a strap according to an embodiment of the present invention.

FIG. 3 is a schematic representation of an embodiment of a non-extended strap including a cam plate.

FIG. 4 is a schematic representation of an embodiment of a partially extended strap including a cam plate.

FIG. 5 is a schematic representation of an embodiment of a fully extended strap including a cam plate.

FIG. 6 is a graph showing a spring rate profile in a strap according to an embodiment of the present invention.

FIG. 7 is a graph showing a further spring rate profile in a strap according to an embodiment of the present invention.

FIG. 8 is a perspective drawing representing an embodiment of a strap with a cam plate seen from the front of the cam plate, according to an embodiment of the present invention.

FIG. 9 is a perspective drawing of the strap of FIG. 8, as seen from the back of the cam plate.

FIG. 10 is a schematic representation of an embodiment of a strap with a material having a distributed mechanism that exhibits a semi-flat spring rate.

FIG. 11 is a schematic representation of the changes in an element of the mechanism in FIG. 10 as the strap material is extended.

FIG. 12 is a schematic sectional representation of the mechanism of FIG. 10 positioned between two rubber-like sheets, seen from the side.

FIG. 13 is a schematic representation of another embodiment of an element of semi-flat spring rate strap material.

FIG. 14 is a schematic sectional representation of a semi-flat spring rate strap mechanism seen from the side, according to a further embodiment of the present invention.

FIG. 15 is a perspective drawing showing a cover for semi-flat spring rate strap gear, as seen from the inside, according to an embodiment of the present invention.

FIG. 16 is a further perspective view of the cover of FIG. 15.

FIG. 17 is a schematic representation of a further embodiment of the invention incorporating an array of elements that move outwards as the strap is stretched.

### DESCRIPTION

### A. Glossary

The term “degressive” in relation to a spring means that it has a spring rate that is declining as the extension of the spring is increasing. The force to maintain the spring extended further from a given point may be greater or lower than that at the given point, but if it is greater it is not as high as to be proportional to the additional extension of the spring (e.g. curve 79, FIG. 7).

The terms “inelastic” and “non-elastic” refer to the non-stretchable property of materials that are not normally considered to be elastic, such as the leather in a belt. While such materials may scientifically exhibit a very small and negligible amount of elasticity, they are still considered to be inelastic. Generally, leather or polymer is multiple orders of magnitude stiffer than an elastic component.

The term “spring rate” of a linear spring refers to the spring constant k given by Hooke's law F=kx, where F is the applied force and x is the extension of the spring.

The term “spring rate profile” refers to a graph of force (or tension) on the y-axis against extension along the x-axis for springs that do not obey Hooke's law, and therefore are not linear springs.

The term “semi-flat” refers to a spring rate profile that is typically flat, approximately flat or degressive over a particular extension range of interest of the spring. In some cases, the spring rate profile may even be slightly progressive, but the minimum requirement for the spring rate profile to be semi-flat is that the percentage variation in extension force is smaller, by at least 10%, than the percentage variation in extension over a range of extension of interest, such as an operating range.

### B. Overview

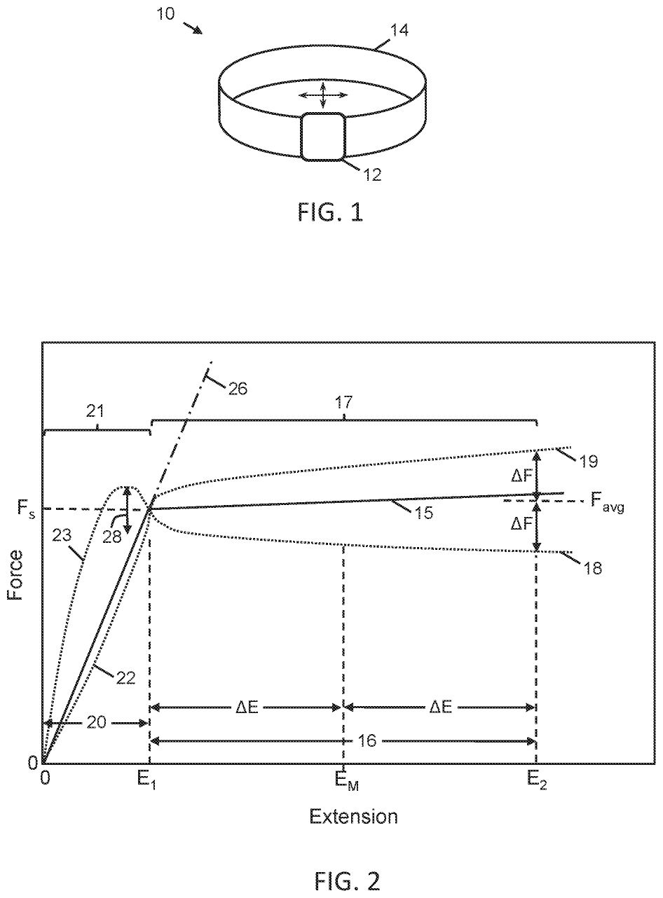

Referring to FIG. 1, a representation of a strap 10 is shown having a central piece 12 connected to a band 14 to form a loop, which is fastened around an expandable object, such as a user's leg, for example. In some embodiments, the strap 10 is either connected to or through a device or object that is to be held onto the user's leg. In some embodiments, the object or device forms part of the strap 10.

In one embodiment the band 14 exhibits an inelastic behavior while the central piece 12 has a semi-flat spring rate over a major portion of its operating range, i.e. a spring rate that is flat, degressive or otherwise allows for a relatively wide range of extension with a relatively small variation in force. Using a semi-flat spring rate helps to prevent an increased force to human body parts such as legs, waist, arms or torso as those body parts expand.

In another embodiment the central piece 12 exhibits an inelastic behavior while the band 14 has a semi-flat spring rate over a major portion of its operating range.

Thus, the present invention can be represented as a strap 10 having a combination of at least two portions, a first portion that has non-elastic behavior and a second portion that has a semi-flat spring rate. As a consequence, when the strap 10 is coupled to a human leg and a movement, such as a squat, calls for an increased extension of the strap, the additional pressure on the leg due to the strap extension is minimized, preventing excessive discomfort to the wearer.

FIG. 2 shows an example of the spring rate profile of the strap 10. The spring rate profile 15 has an operating range of interest 16, which extends from a first extension E₁ to a second extension E₂. The mid-point of the working range is E_{M} and the working range can be expressed as E_{M}±ΔE. In use the strap 10 is fitted to a person such that the range of extension of the strap as a result of movements of the person lies within the operating range of interest 16. In the operating range of interest 16, the spring rate profile is semi-flat, as shown by portion 17 of the profile 15. Dotted lines 18, 19 show alternate spring rate profiles for the operating range of interest 16. The acceptable variation of spring force in the operating range of interest 16 is shown as ±ΔF relative to average force F_{avg}.

The strap also has an initial extension range 20 for pre-tensioning the strap. In the initial range 20, the spring rate profile 21 is linear, progressive, degressive or a combination of these, and is steep compared to the profile 17 in the operating range of interest 16. Force Fₛ is needed to set the spring into its working range. Dotted lines 22, 23 show exemplary extents of other spring rate profiles in the initial range 20. The result of using such a spring rate profile 15 is that a relatively large force is needed to prime or preload the strap 10, by stretching it beyond a certain minimum extension E₁ but then not much additional force, if any, is needed to continue to extend the strap over its full operating range 16 up to extension point E₂. In contrast, if a linear spring rate spring rate 26 were used for the operating range, the force would increase excessively in comparison, causing considerable discomfort to the user.

In most embodiments, the semi-flat spring rate profile is such that the variation in extension force ±ΔF/F_{avg} is smaller, by at least 25%, than the corresponding variation in extension ±ΔE/E_{M} within the operating range.

Range 28 represents an extra-tight force region in the initial range 21 of the spring rate profile 23, which must be overcome for the strap to be properly pre-tensioned. This provides a physical indication or a feel to the user that the strap is set correctly for use. The range 28 can be changed in relative height depending on the embodiment.

In FIG. 2, as in all other graphs and throughout this document, the x-axis is extension and the y-axis is force or tension, such that steeper slopes represent stronger linear springs and shallow slopes represent relatively weaker linear springs.

### C. First Exemplary Embodiment

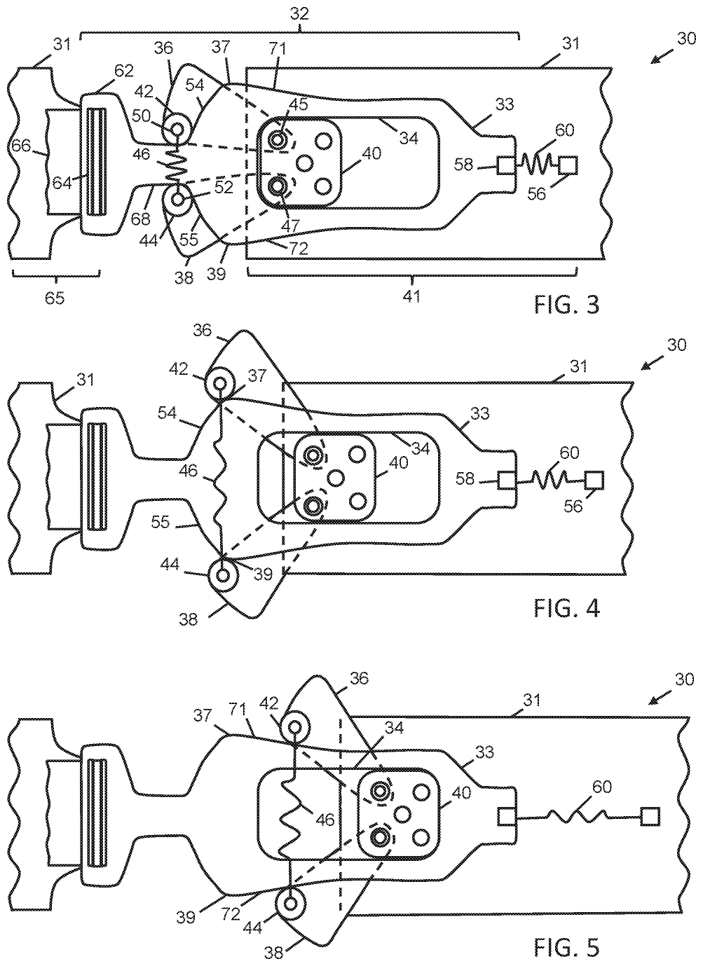

FIG. 3 shows an embodiment of a non-extended strap 30 with an inelastic band 31 and cam mechanism 32, corresponding to the band 14 and central piece 12 of FIG. 1.

The cam mechanism 32 has a cam plate 33, a guide 34 defined by the cam plate, two or more followers 36, 38 and a slider 40. The slider 40 is connected non-adjustably to an end region 41 of the band 31 and slides in the guide 34 as the strap 30 is extended, i.e. by pulling the band to the right relative to the cam mechanism 32. The followers 36, 38 each include a wheel 42, 44 mounted thereon, wherein wheel 42 is connected to wheel 44 via a coil spring 46. The followers 36, 38 are biased towards each other by the coil spring 46, or, in other embodiments, another type of spring. The followers 36, 38 are shown behind the cam plate 33, however, duplicate follower plates (not shown) are also present over the cam plate, such that the wheels 42, 44 are each sandwiched between two follower plates. The followers 36, 38 are pivoted at points 45, 47 on the slider 40. Extremities of the coil spring 46 are attached respectively to pivots 50 and 52 located at the center of each wheel 42, 44. Rounded corners 37 and 39 are present in the cam plate 33, between the shoulders 54, 55 and the sides 71, 72 of the cam plate 33. The combination of a shoulder 54, rounded corner 37 and side 71 can be referred to as a profiled edge or cam of the cam plate.

In this view, the strap 30 is not extended, thus the slider 40 is positioned at the left of the guide 34. When the strap 30 is not in extension, the rotating wheel 42 of the follower 36 is located at the inside of the shoulder 54 of the cam plate 33, in contact with the neck 68 of the cam plate 33. Also, the rotating wheel 44 of the follower 38 is located at the inside of the other shoulder 55 of the cam plate 33, also in contact with the neck 68.

The band 31 is attached indirectly to the cam plate 33 via a fixation piece 56 located on the surface of the band. The fixation piece 56 is connected to another fixation piece 58 located on the cam plate 33 by means of a coil spring 60 or other sprung connection. The other end region 65 of the band 31 is connected to a buckle 62 (or other adjustable, disconnectable attachment) at the head of the cam plate 33. The end 66 of the band 31 is narrower than the main part of the band in order to slide inside the holes 64 of the buckle 62.

Instead of the buckle 62, other fixing mechanisms can be used in other embodiments, such as a ratchet strap, Velcro®, a Boa® system, etc.

The coil spring 46, between the wheel 42 located on the follower 36 and wheel 44 of the other follower 38, is at its minimum extension, across a narrow portion or neck 68 of the cam plate 33.

FIG. 4 represents the same embodiment as in FIG. 3 but this time the strap 30 is partially extended. When partially extended, the band 31 pulls the slider 40 to the right relative to the guide 34, the slider 40 moving inside the guide 34. The motion of the slider 40 is resisted by the action of the followers 36, 38, connected by the coil spring 46, being forced outwards along the shoulders 54, 55 of the cam plate 33. The wheels 42, 44 of followers 36, 38 are shown positioned on the corners 37, 39 of the cam plate 33. When the wheels 42, 44 are positioned on the corners 37, 39 of the cam plate 33, the extension of the coil spring 46 between the two wheels 42, 44 reaches its maximum value.

When the strap 30 is partially extended, the band 31 pulls the fixation piece 56 to the right relative to the cam plate 33, in turn extending the coil spring 60 that is attached to the fixation piece 58 located on the cam plate. Therefore, the strap 30 is being extended against the combined resisting forces of both springs 46, 60, and the components of the cam plate mechanism 32 interact with each other to provide an overall non-linear spring force profile to the strap 30.

Referring to FIG. 5, the same embodiment of a strap 30 is shown as in FIGS. 3 and 4. In this view, the strap 30 is fully extended and the slider 40 has moved to the right of the guide 34. The wheels 42, 44 of the followers 36, 38 have now rolled over onto the sides 71, 72 of the cam plate 33, past the corners 37, 39. As such, the extension of the coil spring 46 between the two wheels 42, 44 has decreased compared to when the two wheels were located on the corners 37, 39 of the cam plate 33.

The sides 71, 72 are further apart at the rounded corners 37, 39 than they are at locations in contact with the cam followers 36, 38 when the slider 40 is at its maximum travel, as shown in FIG. 5. The decreasing spring rate profile due to the cam followers 36, 38 and sides 71, 72 being angled inwards therefore compensates, at least to some extent, the linear spring rate of the spring 60, leading to a semi-flat spring rate profile when the followers are on the sides of the cam plate. The profiled edges (cams), spring 46 and spring 60 define the spring rate profile of the strap 30.

In use, the non-elastic band 31 is reasonably tightly wrapped around a leg to hold a device in place, and buckled to the cam plate 33. The strap is tensioned by the cam mechanism 32, which provides a declining spring rate due to the shoulders 54, 55, corners 37, 38 and sides 71, 72 of the cam plate 33. Depending on the embodiment one can achieve a flat spring rate or a decreasing spring rate by altering the shapes and/or sizes of the shoulders 54, 55, corners 37, 38 and sides 71, 72 of the cam plate 33, and by changing the spring rates of the coil springs 46, 60.

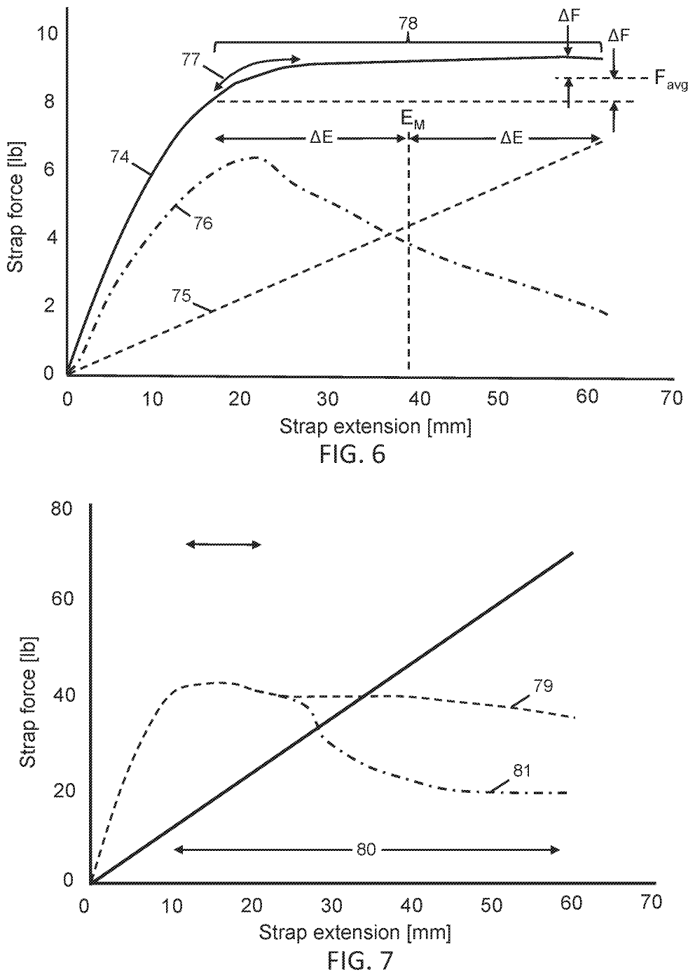

Referring to FIG. 6, a graph is shown depicting the profile 74 of the spring rate of a strap similar to the first embodiment 30 as it undergoes stress. The spring rate profile 74 of the strap 30 can be seen as the sum of two other spring rates 75, 76 of two different components of the strap, namely coil spring 60 and cam mechanism 32 respectively.

The spring rate 75 has a linear behavior in that the force applied to the spring is proportional to its extension. The spring rate profile 76 of the cam mechanism 32 has a degressive behavior. In the first part of the spring rate profile 76, the force increases as the cam mechanism 32 is extended until it reaches about 16 mm of extension. Then, in the second part of the profile, between an extension of about 16 mm and 60 mm, the spring rate profile 76 of the cam mechanism 32 becomes semi-flat, which means that the force applied to the cam mechanism changes little when its extension increases, i.e. there is only a relatively little additional force opposing further extension. In the second part, the force applied on the cam mechanism 32 is relatively stable compared to in the first part of the spring rate profile.

The spring rate profile 74 of the strap as a whole can be seen as the summation of the constant spring rate 75 of the coil spring 60 and the degressive spring rate 76 of the cam mechanism 32.

In this embodiment, the strap 30 must be extended by about 16 mm to prime the strap. In other embodiments, the extension to prime the strap can be smaller than that shown here. The range 77 is the target range (for example) in which the strap is to be used during normal walking, i.e. somewhere in the range of 6-10 lbs (2.7-4.5 kg) tension. During a squat, the extension of the strap extends to about 50 mm. It is the operating range 78, in the spring rate profile 74, that can be defined as the portion that is responsible for mitigating the additional pressure applied on the part of the body to which the strap is fastened, during movements that cause the strap to expand. In this particular embodiment, it can be seen that the force exerted by the strap in the operating range is within the range F_{avg}±ΔF, for an extension range of at least E_{M}±ΔE, where ΔF≤0.15F_{avg} and ΔE=0.55E_{M} where F_{avg} is the average force of the strap in its operating range and E_{M} is the midpoint of the operating range.

Referring to FIG. 7, a graph of the spring rate profile of two other embodiments is shown. In one case 79, the semi-flat portion of the spring rate is set to provide a force of about 401b (18 kg) in the operating range 80. In the other case 81, the semi-flat portion of the spring rate is set to provide a force of about 30±10 lb (13±5 kg) in the operating range 80. During normal walking, 10-20 mm of extension of the strap is expected, and about 18 kg of tension is required. During squats, the strap length is required to grow to 50-60 mm in many cases, however, for some people the strap length is expected to increase by 100 mm or even more. During a squat, as little extra tension is tolerable for comfort it would be advantageous to maintain or even reduce the strap tension. The limit of comfort is about 27 kg of strap tension, at which point circulation may be cut off. For narrow straps, 4.5 kg of force would feel uncomfortable. In some embodiments, a strap width of 2.5 cm allows users to comfortably accommodate a maximum tension of 10 kg. The maximum tension should therefore be selected depending on the width of the strap. In contrast, using a purely elastic strap with a constant spring constant, it is not possible to achieve a tight fit during walking and to allow for full squats comfortably. In the embodiments of FIG. 7, ΔF 0.37F_{avg} and ΔE=0.69E_{M}.

For a strap with spring profile 81, the tension in the strap falls monotonically (i.e. continually without ever rising) as the second portion extends from about 17 mm to the maximum operating extension of 58 mm, which is over the majority of the operating range.

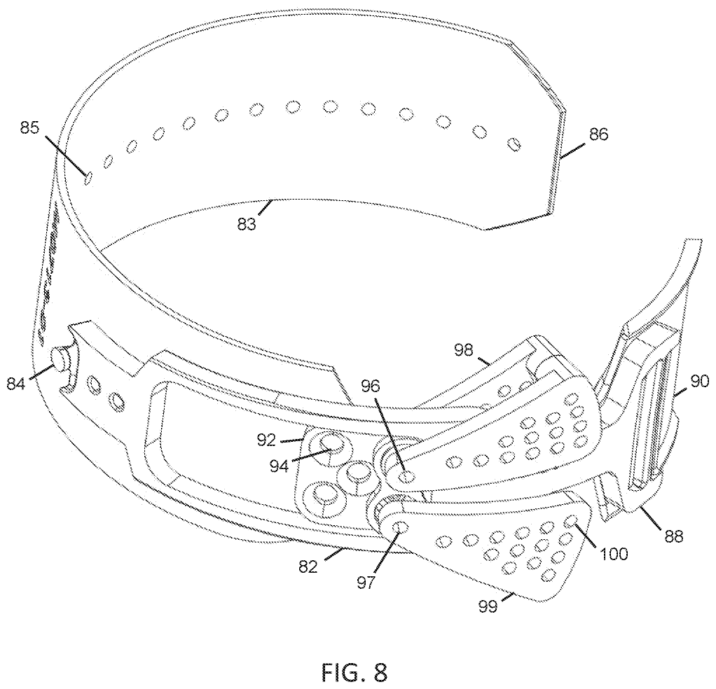

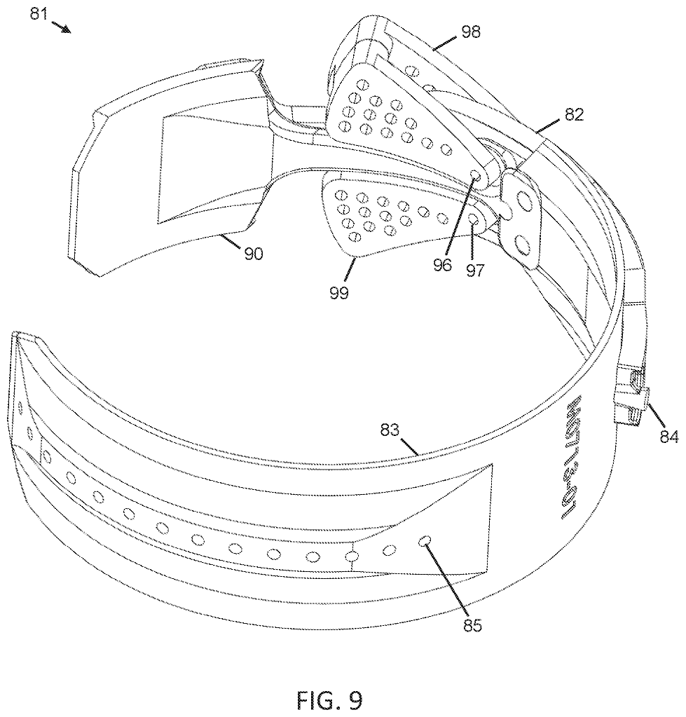

Referring to FIGS. 8 and 9, an embodiment of a strap with a cam plate 82 is shown. An inelastic band 83 is attached to the cam plate 82 indirectly via a coil spring (not shown) and a fixation point 84. The extremity of the band 86 is attached to the cam plate 82 by means of a buckle 88. The extremity of the band 86 passes over a flat guiding piece 90 directly connected to the buckle 88 of the cam plate 82. The slider 92 contains multiple holes 94 for connecting the slider to the band 83. The slider also includes mounting holes that provide connections and pivots 96, 97 for the followers 98, 99. The followers 98, 99 include multiple holes 100 that provide different mounting positions for the spring that biases the followers together. A spring (not shown) is connected between peg 84 and hole 85, or to one of the other holes on band 83 depending on the spring profile required. FIG. 9 illustrates the same strap showing the cam plate 82 from the inside. As it can be seen, the cam plate 82 is curved with a partially cylindrical shape, to correspond more closely to the curvature of a human leg.

### D. Second Exemplary Embodiment

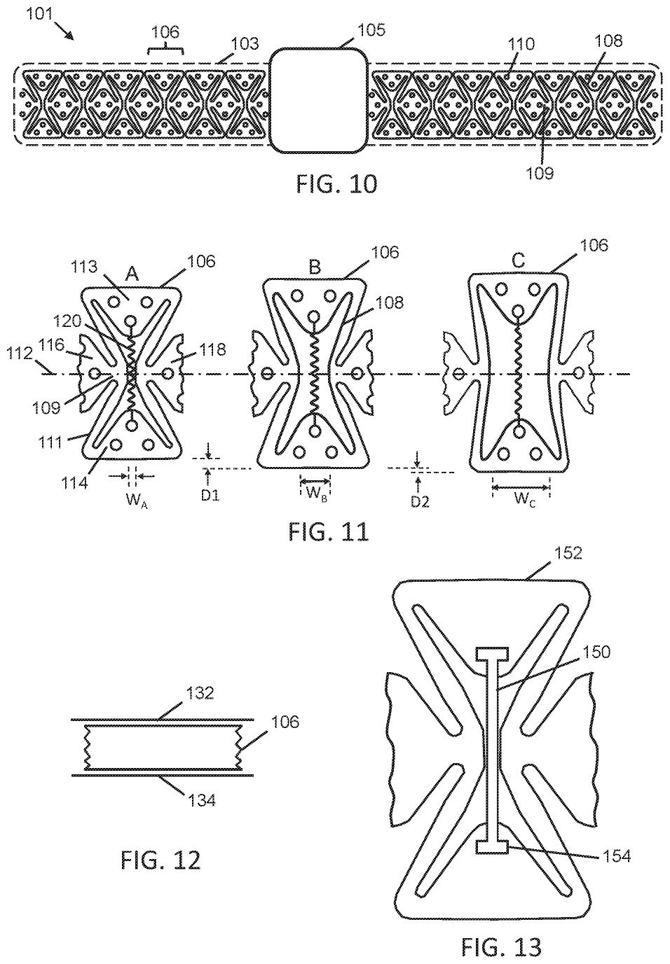

Referring to FIG. 10, another embodiment of strap 101 is shown that has a semi-flat spring rate profile. The strap 101 includes a band 103 and a buckle 105. The buckle 105 is made of a non-elastic material. The band 103 here has a flat or degressive spring rate profile in its operating range. The band is embedded with a succession of elements 106 that may be made of silicone, rubber or other type of flexible material that is shaped to have a non-linear spring rate profile. The structure of an element 106 has, for example, an X-shaped gap 108 or other cut-out, which, combined with the elements being connected to each other at their midpoints 109, provides for a degressive spring rate profile. The structure of an element 106 has, for example, holes 110 for attachment of other components, for ventilation, or for keeping weight to a minimum. This type of strap 101 may be used in order to provide comfort particularly when the strap is worn over thin clothing. This is because the strap grows evenly around the leg during extension, rather than in one relatively small location. It also helps with migration of the strap during use irrespective of the type of clothing worn.

Referring to FIG. 11, an embodiment of an element 106 is shown between rest position A and at two different states of stretch B, C. At A, the element 106 is in its initial, rest position and is not stretched.

At B, the element is stretched out sideways, by a pulling force applied to the midpoints 109 of the sides 111. The pulling force is applied along, or symmetrically in the region of, the centerline 112 of the band 103, to the side regions 116, 118 of the element 106. The width W_{B} at the middle section of the X-shaped gap 108 is increased at B compared to the width W_{A} of the gap when the element 106 is in its rest state at A. The height of the element has also expanded by D1 at both ends. By applying a force to expand the width of the element 106, the resulting increase in height of the element causes a spring 120, which is connected between opposing end regions 113, 114 of the element, to lengthen.

At C, the element 106 is stretched out sideways further and the element increases in height a little further, by D2 at both ends. While the height has only minimally increased from state of stretch B to state of stretch C, the width W_{C} has increased considerably in comparison to width W_{B}. Therefore, as the side regions 116, 118 are pulled apart, the end regions 113, 114 move apart on a declining basis. This corresponds to a degressive spring rate profile since the additional sideways expansion of element 106 incurs a decreasing additional force on the spring 120. At point C, the height of the element 106 has reached its maximum, and so further sideways expansion of the element will result in a lower force from the spring 120 as the height of the element will start to reduce. As well as the spring 120 providing a restoring force to the element, the material of the element itself provides some springiness in some embodiments. The overall spring rate profile of the strap 101 is a sum of the contributions from the material itself and the incorporated springs 120.

Referring to FIG. 12, a sectional view from the side of the band 103 is shown, where element 106 is sandwiched between a sheath of two sheets of material 132, 134. The sheets of material 132, 134 are silicone, for example, and stretch readily as the band 103 is stretched, without contributing significantly to the spring rate profile. Optionally, various degrees of thickness of the material 132, 134 can be chosen depending on the embodiment of the strap. In some embodiments, the material 132, 134 is glued, clipped or otherwise mechanically fastened to the elements 106 at various points. Macro-Velcro® (injection molded little plastic hooks or metal hooks) or rivets are a fastening option as well. In some embodiments, the material 132, 134 provides additional spring force that contributes to the spring rate profile of the strap 101. In particular, the material 132, 134 may be connected to the elements 106 in place of the spring 120. The construction of strap 101 also helps to distribute the stress in a more uniform way over a greater surface of the strap compared to the first embodiment, in which the stress is concentrated in the cam mechanism 32.

In another embodiment, as in FIG. 13, the coil spring is replaced with another elastic component 150 set inside the element 152, such as a rubber strip with enlarged locating ends 154.

### F. Third Exemplary Embodiment

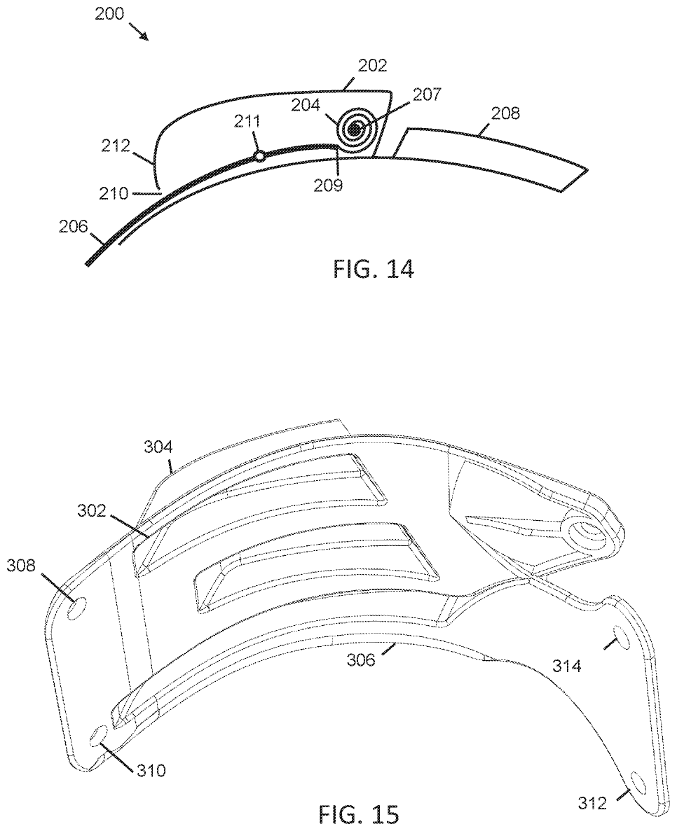

FIG. 14 illustrates schematically another embodiment of a semi-flat spring rate strap 200. This strap 200 is a combination of several elements such as a subshell 202, a flat coil spring 204, a non-elastic band 206 and a buckle 208. The non-elastic band 206 corresponds to band 14 of FIG. 1, and the subshell and flat coil spring 204 to central piece 12. The flat coil spring 204 is attached at its inner extremity 207 to the subshell 202 and at its outer extremity 209 to the non-elastic band 206. The non-elastic band 206 slides inside the subshell 202 via a hole 210 at the front part 212 of the subshell. The non-elastic band 206 has a marker 211 that shows, when aligned with the hole 210 on the subshell 202 or other reference point, that the pre-tensioned or pre-conditioned state of the flat coil spring 204 has been reached (i.e. the strap is primed). The marker may be a notch, a protrusion or a difference in color. The buckle 208 is used to attach and grip the other end of the non-elastic band 206. In some embodiments, an extension (e.g. cables) can be used between the non-linear coil spring 204 and the non-elastic band 206, such that the band 206 remains entirely outside of the subshell 202.

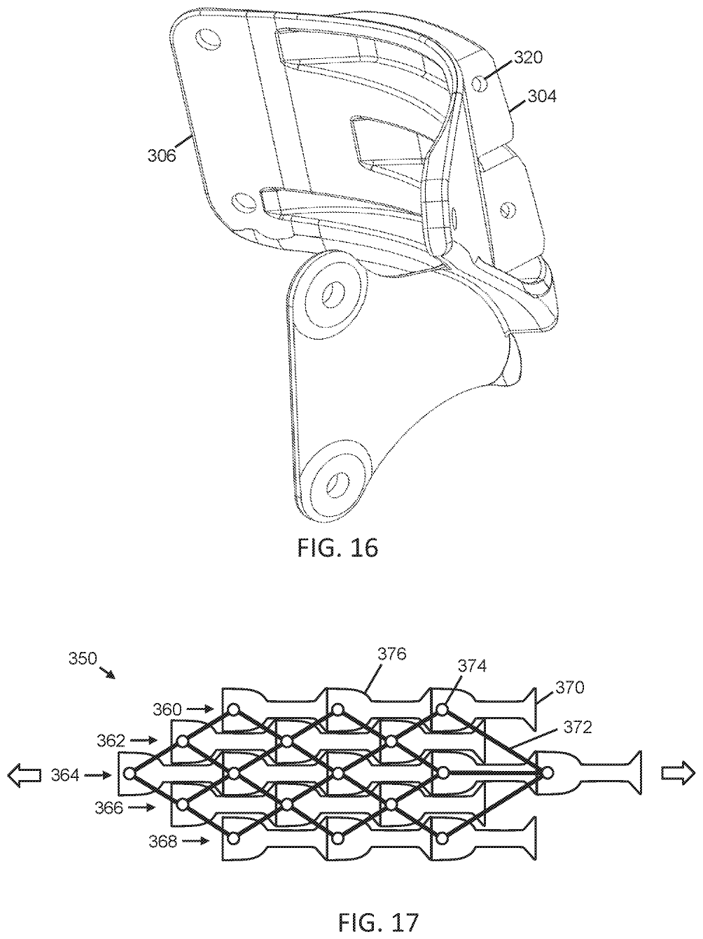

FIGS. 15 and 16 show an illustration of the subshell of the embodiment presented in FIG. 14. One or more hollows 302 in subshell 306 host one or more coil springs (not shown). The hollow 302 is formed by an outward bulge 304 of shell 306. The shell 306 is curved in a way to conform to the morphology of a human limb such as a thigh, i.e. with its inner surface conforming approximately to a partial surface of a cylinder. Holes 308, 310, 312 and 314 located at each extremity of the shell 306 and arranged in pairs are used to fasten an attachment that is intended to keep the band (not shown) in the right position on the shell. For example, the attachment of the band to the shell can be provided by a clipping system mounted using the holes. In FIG. 16, the holes 320 located on the front wall of the bulge 304 of the subshell 306 allow cables to pass through to connect the coil spring (not shown) to the extremity of a band (not shown).

### G. Fourth Exemplary Embodiment

Referring to FIG. 17 a strap with stretchable band 350 includes lengths 360, 362, 364, 366, 368 of individual, rigid “wine glass” shaped elements 370 that are attached with a common elastic mesh 372 at fixing points 374. The elements 370 form an array, in which adjacent elements are offset with respect to each other. As the strap extends, in the direction of the block arrows, the mesh 372 is forced to widen as the wine glass shaped elements 370 separate from each other linearly, and force neighboring elements to move out sideways by their own side profiles 376. The curved side profile 376 of the wine glass elements 370 is tuned to result in a degressive spring rate curve. This construction allows for a more even distribution of the load across the band, and, depending on the materials used, it can help to keep the weight of the strap down.

### G. Variations

While the best presently contemplated mode of carrying out the subject matter disclosed and claimed herein has been presented, other embodiments are possible.

In other embodiments, there is a locking mechanism to lock the cam followers, so that the strap tightening mechanism can be tightened and used as a tourniquet. The cam mechanism has a hard stop that is useful for the tourniquet function.

In some embodiments, the spring rate profile may show hysteresis.

Colored markers may be used in all embodiments to indicate to the wearer that the strap is sufficiently primed. A dial on the cam follower in the first embodiment, which rotates as the strap is extended and aligns with a marker on the housing of the cam, may be used to indicate that the strap is sufficiently primed. In the second embodiment, a hole in the outer layer of the sheath 132 may be positioned to align with a particular coloring on one of the inner elements 106. Other suitable tension indicators may also be employed.

In another embodiment, ΔF≤0.33F_{avg} and ΔE=0.42E_{M} where F_{avg} is the average force of the strap in its operating range and E_{M} is the midpoint of the operating range.

In some embodiments, the strap can be tuned by incorporating adjustment features, such as allowing an initial spring tension to be adjusted by varying its length, or by allowing one spring to be exchanged with another with a different spring rate.

It will be clear to one having skill in the art that further variations to the specific details disclosed herein can be made, resulting in other embodiments that are within the scope of the invention disclosed. All parameters, dimensions, materials, and configurations described herein are examples only and actual values of such depend on the specific embodiment. Accordingly, the scope of the invention is to be construed in accordance with the substance defined by the following claims.

1. A strap for tightening around an expandable object comprising:
a first portion that is inelastic; and
a second portion connected to the first portion and having a spring rate profile that is semi-flat in an operating range spanning a first extension of the second portion and a second extension of the second portion;
wherein, when the strap is tightened around the expandable object, the second portion remains within the operating range as the expandable object expands and contracts.

2. The strap of claim 1, wherein:
tension in the strap in the operating range is in a range F_{avg}±ΔF;
the operating range is E_{M}±ΔE;
ΔF≤0.37F_{avg}, and
ΔE≥0.42E_{M}.

3. The strap of claim 2, wherein the tension in the strap falls monotonically for a majority of the operating range.

4. The strap of claim 1 wherein the spring rate profile is steeper, in a range from zero expansion of the second portion to the first extension of the second portion, than in the operating range.

5. The strap of claim 1, incorporated in a shin brace or a thigh brace of an energy harvester.

6. The strap of claim 1 wherein the first portion is a band and the second portion comprises:
a slider attached to a first end region of the band;
a cam plate with profiled edges and a guide in which the slider is retained and slides;
an adjustable, disconnectable attachment between the cam plate and a second end region of the band;
a sprung connection between the first end region of the band and the cam plate;
a pair of cam followers that are pivoted at the slider, biased towards each other by a spring, and follow the profiled edges as the slider moves along the guide.

7. The strap of claim 6 wherein the cam plate is curved.

8. The strap of claim 6, wherein:
the profiled edges each comprise a shoulder, a side and a rounded corner between the shoulder and side; and
the profiled edges, the spring and the sprung connection define said spring rate profile.

9. The strap of claim 8, wherein the sides are further apart at the rounded corners than they are at locations in contact with the cam followers when the slider is at its maximum travel.

10. The strap of claim 1, wherein the first portion is a buckle and the second portion is a band, the band comprising a plurality of elements, wherein:
adjacent elements are connected to each other via side regions thereof at a centerline of the band;
opposing end regions of each element are biased towards each other by a spring;
each element defines a cut-out that permits side regions of each element to be pulled apart as the band extends;
as the side regions are pulled apart, the end regions move apart on a declining basis.

11. The strap of claim 10, wherein the spring comprises a rubber strip.

12. The spring of claim 10, comprising a layer of flexible material either side of the elements.

13. The spring of claim 10, wherein the cut-out is X-shaped.

14. The strap of claim 1, wherein the first portion is a band and the second portion comprises:
a flat coil spring that is attached between a first end of the band and the second portion;
a marker on the band;
a reference point on the second portion;
wherein when the band is extended so that the marker is beyond the reference point, the strap is in its operating range.

15. The strap of claim 14, wherein the second portion comprises a shell that houses the flat coil spring and is curved in conformity with a curvature of the expandable object.

16. The strap of claim 1, wherein the first portion is a buckle and the second portion is a band, the band comprising an array of adjacent elements, each element connected to a common elastic mesh, wherein:
each element has a side profile that forces two of said elements, which are arranged side by side in an offset relation, to move sideways when the band is extended, thereby widening the array of adjacent elements; and
the elastic mesh is expands sideways as the array of adjacent elements widens.

17. The strap of claim 1, wherein a variation in tension in the strap over the operating range is less than 25% of a variation in extension of the strap over the operating range.
